(19) United States
(12) Patent Application Publication
(10) Pub. No.: US 2016/0089541 A1
(43) Pub. Date: Mar. 31, 2016

(54) DEVICE AND METHOD FOR TREATING A MOVEMENT DISORDER IN A PATIENT

(71) Applicant: Advanced Neuromodulation Systems, Inc. dba St. Jude Neuromodulation Division, Plano, TX (US)

(72) Inventor: Lalit Venkatesan, Prosper, TX (US)

(21) Appl. No.: 14/869,487

(22) Filed: Sep. 29, 2015

Related U.S. Application Data
(60) Provisional application No. 62/056,881, filed on Sep. 29, 2014.

(51) Int. Cl. A61N 1/36; A61N 1/05

(52) U.S. Cl. CPC A61N 1/36185; A61N 1/0529; A61N 1/36067

(57) ABSTRACT

In one embodiment, a neurostimulation system for stimulating neuronal tissue of a brain of a patient, comprises: an implantable pulse generator comprising pulse generating circuitry and a controller; one or more stimulation leads comprising multiple electrodes, the implantable pulse generator adapted to connect to the one or more stimulation leads for delivery of generated electrical pulses to neuronal tissue of the patient; wherein the controller is adapted to control the implantable pulse generator to (i) generate a plurality of bursts of multiple pulses at a frequency of at least 100 Hz and (ii) to deliver each respective bursts on a randomly or pseudo-randomly selected electrode from multiple electrodes of the one or more stimulation leads, wherein a beginning of each burst in the plurality of bursts is separated from a beginning of its respective successive bursts by at least 50 milliseconds.

### RELATED APPLICATION

This application claims the benefit of U.S. Provisional Patent App. Ser. No. 62/056,881, filed Sep. 29, 2014, entitled “DEVICE AND METHOD FOR TREATING A MOVEMENT DISORDER IN A PATIENT,” which is incorporated herein by reference.

### BACKGROUND

Movement disorders refer to a number of conditions including Hypokinesia (Parkinson's disease), Hyperkinetic disorders (L-dopa induced dyskinesia, Hemiballism and Chorea), Dystonia (generalized and localized) and Tremor (Resting, Postural and Action tremor). Parkinson's disease (PD) is a chronic, progressive neurodegenerative movement disorder. The main symptoms are tremors, rigidity, slow movement (bradykinesia), poor balance and difficulty walking. The highest prevalence of PD is in Europe and North America with around 1 to 1.5 million people being affected in the USA. Caucasian populations are affected more than others, with a prevalence of around 120-180 per 100,000 people. Symptoms of PD may appear at any age, but the average age of onset is 60. PD is rare in young people and risk increases with age. The cause of the disease is unknown, but there may be genetic factors.

PD is associated with degeneration of several neuronal modulators in the midbrain that primarily affect the motor system. These include the midbrain dopaminergic nuclei, the serotoninergic median raphe nuclei, the noradrenergic locus coeruleus and the cholinergic pedunculopontine nucleus.

At present, there is no cure for PD. Medical treatment for PD relies on a variety of drugs that stimulate dopamine receptors and although this approach may be effective for 5-10 years, therapy is complicated by motor side effects including “on/off” fluctuations and dyskinesias. With progressive degeneration of the dopaminergic system and other neuronal modulators the patient develops fluctuating responses to medical intervention. Surgery may be contemplated in patients who are poorly controlled on best medical therapy.

Hyperkinetic Disorders are sudden rapid involuntary and purposeless movements that typically intrude into the patient's normal activity. These movements may be both axial and peripheral. Examples of hyperkinetic disorders include L-Dopa dyskinesia, which is a complication of PD, Chorea and Hemiballism, which may result from brain lesions involving the basal ganglia. There are no effective medical treatments for these conditions.

Dystonia is a postural disorder characterized by involuntary muscle contractions affecting various parts of the body including the limbs, trunk, shoulders, face and neck.

Tremor is involuntary oscillatory movements produced by alternating contractions of agonist and antagonist muscles. These movements can affect the proximal and distal limb muscles and also the axial muscle groups. Tremor can occur at rest, with the limb maintained in a particular posture and/or during movements. Tremor can occur as a sign of PD, and as a result of lesions of the basal ganglia, midbrain or the cerebellum, but its most common form is familial Essential Tremor (ET). Medical treatments tend to variably suppress rather than abolish tremor.

At present there are various surgical treatments available for movement disorders; however, many of them involve side effects. Movement disorders are due to abnormal patterns of neuronal firing permeating the motor pathways. Surgical treatment aims to disrupt the transmission of these abnormal patterns by destroying or lesioning motor pathways or nuclei or alternatively overriding the abnormal patterns with high frequency electrical stimulation. The latter treatment is known as Deep Brain Stimulation (DBS) and is achieved by implanting an electrode into the pathways or nuclei in the brain and delivering pulsed electrical current to the tissue from an implanted pulse generator which is connected to the electrode.

A number of targets are known to be effective in the treatment of movement disorders. These include the Globus Pallidus Internus (Gpi), the Ventral Intermediate Nucleus (Vim) of the thalamus and the Subthalamic Nucleus (STN).

Lesions or DBS of the Gpi are effective for the treatment of PD, Dystonia and Hyperkinetic movements. This type of treatment has a modest effect on PD symptoms such as tremor, rigidity, bradykinesia and akinesia, but is effective in treating the motor side effects of L-dopa therapy such as dyskinesia and dystonia which allow the patient to continue on a high dose of medication.

Bilateral Gpi lesions/DBS are associated with worsening axial symptoms including deterioration in speech, swallowing and gait.

Lesions or DBS of the Vim are effective for the treatment of PD tremor but do not affect other symptoms of PD. Typically the Ventralis Intermedius (Vim) nucleus of the thalamus is the target of choice for the treatment of ET. Lesioning is reported to provide good contralateral tremor suppression. However recurrence may occur within weeks or years and long-term studies show that significant tremor persists in 17-32% of cases. Bilateral lesions are associated with significant complications including permanent speech impairment in over 25% and memory and language dysfunction in over 50% of cases.

Clinical studies suggest that DBS of Vim is as effective as lesioning in controlling ET but is likewise associated with side effects, particularly when carried out bilaterally with 30-50% patients suffering from dysarthria and dysequilibrium. However the adverse effects associated with DBS are generally reversible by adjusting the stimulation parameters, though this may be that the expense of satisfactory tremor control. Patients treated with DBS are also reported to develop tolerance (habituation) to stimulation, despite increasing its amplitude. Patients are advised to turn the stimulators “off” at night and take stimulation holidays for weeks, in order to prevent tissue habituation.

Lesioning of the subthalamic nucleus is known to improve tremor, rigidity, bradykinesia and akinesia and allows patients to reduce their medications, which in turn enables patients to reduce their medication. However, the Subthalamic nucleus is a small structure measuring 12 mm anteroposteriorly, 3 mm in width and 6 mm dorso-ventrally; and misplacement of a lesion can cause significant and permanent side effects. As a result, most centers prefer to implant DBS electrodes into the STN because side effects are generally reversible by reducing or stopping stimulation. DBS of the STN is currently the surgical treatment of choice for PD, nevertheless it is not without side-effects. Houeto et al., reported worsening of anxiety and depression following DBS of STN with a prevalence of anxiety in 75% of patients. Bemey et al., reported that DBS of STN can provoke depression in 25% with several having suicidal tendencies. Mania has also been reported. Some groups have, in addition, reported worsening of speech.

In addition to motor functions, the STN has limbic and associative functions. Disruption of these with DBS may contribute to worsening anxiety and depression seen with this treatment. Medial to the STN are fibers carrying cerebellar information to the thalamus and spread of current to these may interfere with information regarding precision movements of the larynx and hence cause worsening of speech. Stimulation of structures anterior and ventral to the subthalamic nucleus including the substantia nigra and area of Sano are associated with severe depression and mania/rage respectively.

### BRIEF DESCRIPTION OF THE DRAWINGS

FIG. 1 depicts a stimulation system for treating a movement disorder according to one representative embodiment.

FIG. 2 depicts a flowchart for treatment of a movement disorder according to one representative embodiment.

FIG. 3 depicts a stimulation pattern of pulses across multiple electrodes disorder according to one representative embodiment.

### DETAILED DESCRIPTION

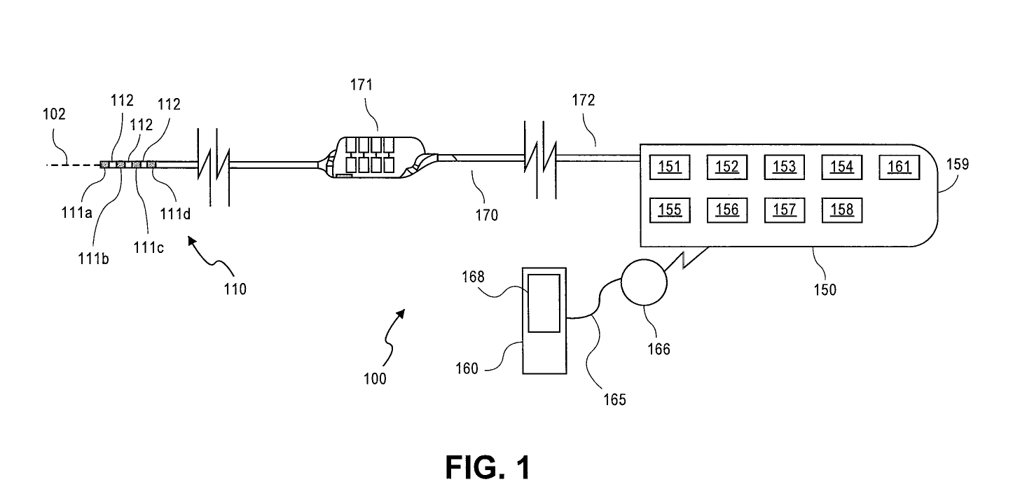

FIG. 1 depicts an NS system 100 for providing a neurostimulation therapy to a patient according to some embodiments of the present disclosure. NS system 100 includes an implantable pulse generator (IPG) 150 that is adapted to generate electrical pulses for application to tissue of a patient. The IPG 150 typically comprises a metallic housing that encloses a controller 151, pulse generating circuitry 152, a charging coil 153, a battery 154, a far-field and/or near field communication circuitry 155, battery charging circuitry 156, switching circuitry 157, and the like. The controller 151 typically includes a microcontroller or other suitable processor for controlling the various other components of the device. Software code is typically stored in memory of the IPG 150 for execution by the microcontroller or processor to control the various components of the device. An example of a suitable IPG is the BRIO™ implantable pulse generator manufactured by St. Jude Medical, Inc.

IPG 150 may comprise a separate or an attached extension component 170. If the extension component 170 is a separate component, the extension component 170 may connect with the “header” portion of the IPG 150 as is known in the art. If the extension component 170 is integrated with the IPG 150, internal electrical connections may be made through respective conductive components. Within the IPG 150, electrical pulses are generated by the pulse generating circuitry 152 and are provided to the switching circuitry 157. The switching circuitry 157 connects to outputs of the IPG 150 (through blocking capacitors). Electrical connectors (e.g., “Bal-Seal” connectors) within the connector portion 171 of the extension component 170 or within the IPG header may be employed to conduct various stimulation pulses. The terminals of one or more leads 110 are inserted within connector portion 171 or within the IPG header for electrical connection with respective connectors. Thereby, the pulses originating from the IPG 150 are provided to the leads 110. The pulses are then conducted through the conductors of the lead 110 and applied to tissue of a patient via stimulation electrodes 111a-d. Any suitable known or later developed design may be employed for connector portion 171.

Stimulation electrodes 111a-d may be in the shape of a ring such that each stimulation electrode 111a-d continuously covers the circumference of the exterior surface of the lead 110. Each of the stimulation electrodes 111a-d are separated by non-conducting material 112, which electrically isolate each stimulation electrode 111a-d from an adjacent stimulation electrode 111a-d. The non-conducting material 112 may include one or more insulative materials and/or biocompatible materials to allow the lead 110 to be implantable within the patient. The stimulation electrodes 111a-d may be configured to emit the pulses in an outward radial direction proximate to or within a stimulation target. Additionally or alternatively, the stimulation electrodes 111a-d may be in the shape of a split or non-continuous ring such that the pulse may be directed in an outward radial direction adjacent to the stimulation electrodes 111a-d. Multiple such “segmented” electrodes may be disposed at a given longitudinal position along lead 110 to more finely control application of pulses to one or more neural population(s) during therapeutic operations of NS system 100. Examples of a fabrication process of the stimulation electrodes 111a-d is disclosed in U.S. patent application Ser. No. 12/895,096, entitled, “METHOD OF FABRICATING STIMULATION LEAD FOR APPLYING ELECTRICAL STIMULATION TO TISSUE OF A PATIENT,” which is expressly incorporated herein by reference.

The lead 110 may comprise a lead body 172 of insulative material about a plurality of conductors within the material that extend from a proximal end of lead 110, proximate to the IPG 150, to its distal end. The conductors electrically couple a plurality of the stimulation electrodes 111a-d to a plurality of terminals (not shown) of the lead 110. The terminals are adapted to receive electrical pulses and the stimulation electrodes 111a-d are adapted to apply the pulses to the stimulation target of the patient. Also, sensing of physiological signals may occur through the stimulation electrodes 111, the conductors, and the terminals. It should be noted that although the lead 110 is depicted with four stimulation electrodes 111a-d, the lead 110 may include any suitable number of stimulation electrodes 111a-d (e.g., less than four, more than four) as well as terminals, and internal conductors. Additionally or alternatively, various sensors may be located near the distal end of the lead 110 and electrically coupled to terminals through conductors within the lead body 172.

For implementation of the components within the IPG 150, a processor and associated charge control circuitry for an IPG is described in U.S. Pat. No. 7,571,007, entitled “SYSTEMS AND METHODS FOR USE IN PULSE GENERATION,” which is expressly incorporated herein by reference. Circuitry for recharging a rechargeable battery (e.g., battery charging circuitry 156) of an IPG using inductive coupling and external charging circuits are described in U.S. Pat. No. 7,212,110, entitled “IMPLANTABLE DEVICE AND SYSTEM FOR WIRELESS COMMUNICATION,” which is expressly incorporated herein by reference.

An example and discussion of “constant current” pulse generating circuitry (e.g., pulse generating circuitry 152) is provided in U.S. Patent Publication No. 2006/0170486 entitled “PULSE GENERATOR HAVING AN EFFICIENT FRACTIONAL VOLTAGE CONVERTER AND METHOD OF USE,” which is expressly incorporated herein by reference. One or multiple sets of such circuitry may be provided within the IPG 150. Different pulses on different stimulation electrodes 111a-d may be generated using a single set of the pulse generating circuitry 152 using consecutively generated pulses according to a “multi-stimset program” as is known in the art. Complex pulse parameters may be employed such as those described in U.S. Pat. No. 7,228,179, entitled “Method and apparatus for providing complex tissue stimulation patterns,” and International Patent Publication Number WO 2001/093953 A1, entitled “NEUROMODULATION THERAPY SYSTEM,” which are expressly incorporated herein by reference. Alternatively, multiple independent current sources may be employed to provide pulse patterns (e.g., tonic stimulation waveform, burst stimulation waveform) that include generated and delivered stimulation pulses through various stimulation electrodes of one or more leads 111a-d as is also known in the art. Various sets of parameters may define the pulse characteristics and pulse timing for the pulses applied to the various stimulation electrodes 111a-d as is known in the art. Although constant current pulse generating circuitry is contemplated for some embodiments, any other suitable type of pulse generating circuitry may be employed such as constant voltage pulse generating circuitry.

Controller device 160 may be implemented to charge/recharge the battery 154 of the IPG 150 (although a separate recharging device could alternatively be employed) and to program the IPG 150 on the pulse specifications while implanted within the patient. Although, in alternative embodiments separate programmer devices may be employed for charging and/or programming the NS system 100 and far-field communication may be employed. The controller device 160 may be a processor-based system that possesses wireless communication capabilities. Software may be stored within a non-transitory memory of the controller device 160, which may be executed by the processor to control the various operations of the controller device 160. A “wand” 138 may be electrically connected to the controller device 116 through suitable electrical connectors (not shown). The electrical connectors may be electrically connected to a telemetry component 166 (e.g., inductor coil, RF transceiver) at the distal end of wand 138 through respective wires (not shown) allowing bi-directional communication with the IPG 150. Optionally, in some embodiments, the wand 138 may comprise one or more temperature sensors for use during charging operations. In other embodiments, far field communication circuitry may also be employed to communicate data between IPG 150 and controller device 160.

The user may initiate communication with the IPG 150 by placing the wand 138 proximate to the NS system 104. Preferably, the placement of the wand 138 allows the telemetry system of the wand 138 to be aligned with the far-field and/or near field communication circuitry 155 of the IPG 150. The controller device 160 preferably provides one or more user interfaces 168 (e.g., touchscreen, keyboard, mouse, buttons, or the like) allowing the user to operate the IPG 150. The controller device 160 may be controlled by the user (e.g., doctor, clinician) through the user interface 168 allowing the user to interact with the IPG 150. The user interface 168 may permit the user to move electrical stimulation along and/or across one or more of the lead(s) 110 using different stimulation electrode 111a-d combinations.

Also, the controller device 160 may permit operation of the IPG 150 according to one or more stimulation programs to treat the patient. Each stimulation program may include one or more sets of stimulation parameters of the pulse including pulse amplitude, pulse width, pulse frequency or inter-pulse period, pulse repetition parameter (e.g., number of times for a given pulse to be repeated for respective stimset during execution of program), biphasic pulses, monophasic pulses, etc. The IPG 150 modifies its internal parameters in response to the control signals from the controller device 160 to vary the stimulation characteristics of the stimulation pulses transmitted through the lead 110 to the tissue of the patient. NS systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are expressly incorporated herein by reference.

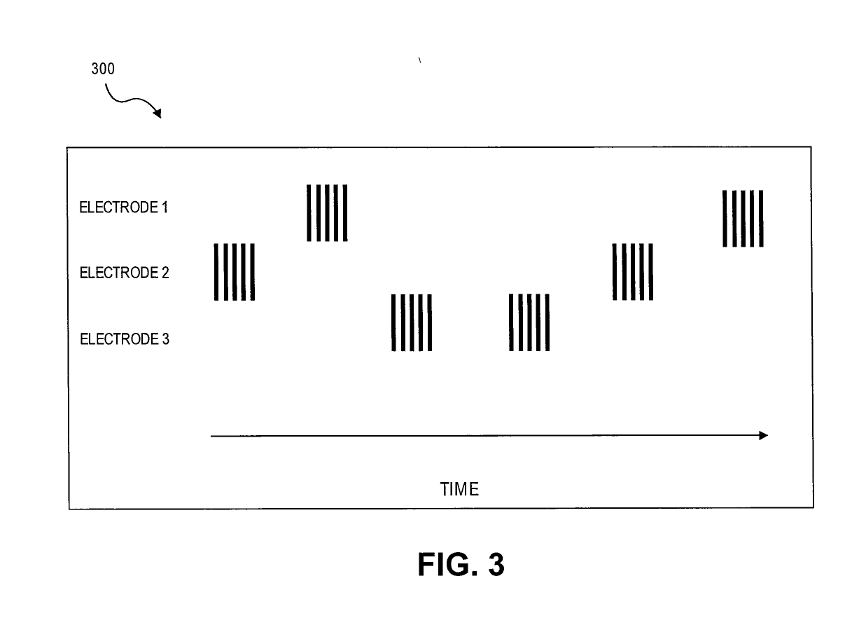

According to embodiments described herein, NS system 100 is adapted to treat a movement disorder in a patient by delivering bursts of pulses through multiple electrodes of a stimulation lead. The electrodes are implanted within a suitable target location in the patient's brain (for example, within or near the STN). Preferably, the order of deliver of bursts on the respective electrodes occurs on a random or pseudo-random basis. For example, a suitable random number generation algorithm (e.g., the yarrow algorithm and the fortuna algorithm) may be employed to select a given electrode. Preferably but not critically, the probability of selection of electrodes among the set of available is mostly uniform, although mathematically rigorous random probability is not required. A limited sequence of stored pseudo-random numbers, hash based algorithm, or linear shift feedback generator implementation may also be suitably employed. A burst of stimulation pulses is delivered via the selected electrode. A suitable delay is applied in which no stimulation pulses are applied to neuronal tissue. Then, the process is repeated by selection of another electrode. Preferably, at least three electrodes are selected for the random delivery of stimulation pulse bursts. FIG. 3 depicts example stimulation pattern 300 across three electrodes of a stimulation lead according to one representative embodiment. In some embodiment implement, an IPG employs multiple “stim sets” to generate electrical pulses as, for example, described in U.S. Pat. No. 7,228,179. Different stim sets for the IPG are programmed in such a fashion that each stim set delivers the stimulation output through different electrode contacts. Then, the IPG is programmed to randomize activation of the respective stim sets.

In some representative embodiments, each burst comprises a suitable number of pulses ranging (e.g., four (4) or more pulses). The repetition rate for pulses within a given pulse is preferably selected to be at least 100 Hz and up to 240 Hz. The amplitude and pulse width of the pulses are preferably selected according to conventional deep brain stimulation methodologies (e.g., amplitudes from 0.05-12.75 mA and pulses widths between 50-150 μs). The amplitude and pulse width parameters may be individually selected for each electrode of the stimulation lead. The amplitude and pulse parameters may be selected to optimize management of movement disorder symptoms while avoiding, reducing, or otherwise limiting undesirable side effects. In one embodiment, an amplitude of 0.2 mA is selected.

In one specific embodiment, each burst contains five pulses repeated at a pulse rate of 130 Hz. A delay period is applied after the end of each burst equal to 20 milliseconds or more. Preferably, the beginning point of a first burst occurs at least 50 milliseconds before the beginning of the next subsequent burst in this stimulation therapy. Also, the pulses are provided to the patient for a limited duration during a given day. For example, the stimulation pattern may be provided for approximately 2 hours to approximately 4 hours per day.

It is believed that the stimulation protocol described herein provides patients with an effective therapy for management of movement disorders. The stimulation protocols described herein involve less power consumption as compared to certain other known stimulation protocols. Further, the stimulation protocols described herein may lessen side effects by reducing the amount of time that stimulation is provided to the patient. Specifically, it is believed that the stimulation protocols described herein may generate a “carry-over” effect where movement disorder symptoms are managed even when stimulation is not provided to the patient based upon data related to use of such stimulation protocols with the 1-methyl-4-phenyl-1,2,3,6-tetrahydropyridine (MPTP) monkey model of PD.

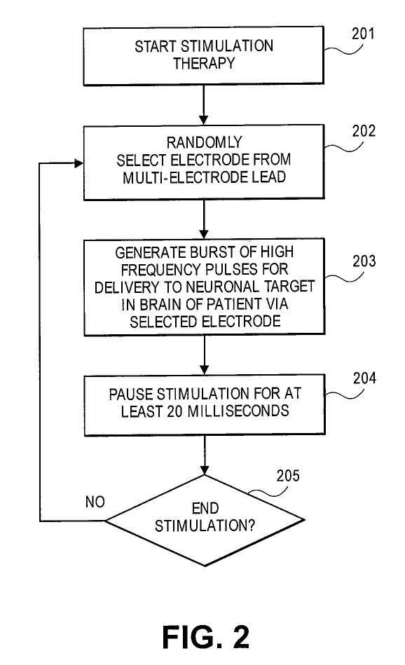

FIG. 2 depicts a stimulation protocol according to one representative embodiment. In 201, the stimulation therapy starts. In 202, an electrode from multi-electrode lead is randomly selected. In 203, a burst of high frequency pulses is generated by an IPG implanted in the patient for delivery to neuronal target in brain of patient via selected electrode. In 204, the stimulation is paused for at least 20 milliseconds. In 204, a logical determination is made (in the implantable pulse generator) to determine whether to end stimulation. For example, the stimulation therapy may be continued for 2 to 4 hours. If not, the process is repeated beginning at 202.

The controllers and devices discussed herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the controllers and devices discussed herein may include circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The controllers and devices discussed herein may execute a set of instructions that are stored in one or more storage elements, in order to process data. The storage elements may also store data or other information as desired or needed. The storage element may be in the form of an information source or a physical memory element within the controllers and devices discussed herein. The set of instructions may include various commands to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f), unless and until such claim limitations expressly use the phrase “means for” followed by a statement of function void of further structure.

1. A neurostimulation system for stimulating neuronal tissue of a brain of a patient, comprising:
an implantable pulse generator comprising pulse generating circuitry and a controller;
one or more stimulation leads comprising multiple electrodes, the implantable pulse generator adapted to connect to the one or more stimulation leads for delivery of generated electrical pulses to neuronal tissue of the patient;
wherein the controller is adapted to control the implantable pulse generator to (i) generate a plurality of bursts of multiple pulses at a frequency of at least 100 Hz and (ii) to deliver each respective bursts on a randomly or pseudo-randomly selected electrode from multiple electrodes of the one or more stimulation leads, wherein a beginning of each burst in the plurality of bursts is separated from a beginning of its respective successive bursts by at least 50 milliseconds.

2. The neurostimulation system of claim 1 wherein the controller repeats pulses within each burst at a rate of at least 130 Hz.

3. The neurostimulation system of claim 1 wherein the controller pseudo-randomly selects between at least three electrodes for delivery of generated pulses to tissue of the patient.

4. The neurostimulation system of claim 1 wherein the controller selects electrodes according to relatively equal probability.

5. The neurostimulation system of claim 1 wherein pulses in the plurality of bursts are constant current pulses.

6. The neurostimulation system of claim 5 wherein pulses in the plurality of bursts are at an amplitude of 0.2 mA.

7. The neurostimulation system of claim 1 wherein the controller waits an amount of time between generation of respective bursts according to a delay parameter.

8. The neurostimulation system of claim 7 wherein the delay parameter is 20 milliseconds.

9. The neurostimulation system of claim 7 wherein the controller randomly selects one of multiple sets of stimulation parameters for activation for generation of electrical pulses on a random or pseudo-random basis.
